(19) United States
(12) Patent Application Publication
(10) Pub. No.: US 2017/0368318 A2
(43) Pub. Date: Dec. 28, 2017

(54) ANGIOPLASTY DEVICE

(71) Applicant: Amram ASHER, Sitriya (IL)

(72) Inventor: Amram ASHER, Sitriya (IL)

(21) Appl. No.: 15/360,009

(22) Filed: Nov. 23, 2016

(51) Int. Cl. A61M 25/10

(52) U.S. Cl. CPC A61M 25/104; A61M 25/1002; A61M 2025/105; A61M 2025/1079; A61M 2025/1063

(57) ABSTRACT

Provided is a catheter system for intraluminal passages, including blood vessels, which includes a guidewire incorporating a work element, and an inflatable balloon enveloping the work element. The work element includes a lesion-smoothing member which in a work configuration projects radially from a longitudinal axis of the balloon. The work element may be configured to allow for controllable rotation about the longitudinal axis, such that the rotation thereby substantially defines a closed ellipsoid-like surface. When the balloon is inflated with a fluid and is inside an intraluminal passage, rotation of the work element causes the lesion-smoothing member to rotate along a surface of the balloon and simultaneously push against the balloon surface. The pushing against the balloon surface exerts force against the walls of the intraluminal passage, thereby allowing for smoothing lesion material located on the walls of the intraluminal passage.

### FIELD OF THE INVENTION

The invention, in some embodiments, relates to the field of balloon catheters and more particularly, but not exclusively, to intravascular balloon catheters.

### BACKGROUND OF THE INVENTION

Stenosis, or the narrowing of a blood vessel lumen due to the formation of a lesion (e.g. deposits such as cholesterol, fats, and calcium) is a medical condition common in both women and men over 50. In some cases, such as in a resultant formation of a blood clot in a coronary artery, the condition may turn life-threatening.

One standard technique for the removal of a lesion in a blood vessel lumen is balloon angioplasty. A balloon catheter is inserted into the blood vessel and guided therein until the balloon is adjacent to the lesion. The balloon is then inflated, thereby compressing the lesion.

A related technique is that of stenting. A stent carried on the balloon is expanded against the blood vessel walls as the balloon is inflated. When the balloon is deflated and pulled out, due to the stent's plasticity, the stent remains in place and prevents the blood vessel from reassuming its original shape (e.g. decompressing). Drawbacks include the risk of formation of blood clots, and the growth of scar tissue due to an immune response to the foreign body (the stent).

Other techniques include atherectomy, the insertion of a catheter including blades, which are used to cut up the lesion, and laser ablation, the insertion of a catheter including, for example, a laser heated optical fiber tip, which is used to disintegrate the lesion. As compared to balloon angioplasty, both of the latter techniques have the drawback of a higher risk of damaging the blood vessel walls.

Balloon angioplasty is also not devoid of drawbacks. In fact, about a third of patients undergoing angioplasty will return for a second angioplasty within six months due to a recurrence of stricture (restenosis). In standard balloon angioplasty, once on site, the balloon is inflated with fluid, resulting in a substantially equal pressure throughout the balloon, and, in particular, in substantially equal pressure being applied to the blood vessel walls. The lesion, however, will generally not be uniformly stiff, and will therefore not be uniformly compressed. This lack of uniform compression may lead to an undesirable on-site reshaping of the blood vessel walls, whereby their surfaces become wavelike (instead of smooth), increasing the chances of restenosis.

WO 2013/080213 to Teichman and Kotlizky discloses a balloon catheter system. The catheter includes a first balloon disposed around a second balloon which is movable within the first balloon.

U.S. Pat. No. 4,338,942 to Fogarty discloses a dilatation catheter with a double lumen tube and inner and outer inflatable and deflatable balloon elements, one within the other. The inner bag element is twisted for retraction while the outer bag element is inflated. Subsequent deflation of the outer bag element serves to further laterally compress the inner bag element and provide a smooth buffering surface for engagement with blood vessel walls as the catheter is moved past them.

There remains a need for improved methods for smoothing lesion material on a blood vessel wall without leading to adverse effects.

### SUMMARY OF THE INVENTION

To overcome the shortcomings listed above, there is disclosed herein a balloon catheter including a work element housed within an inflatable balloon. The work element is configured to allow applying an outward force on a target area on a surface of the balloon when the balloon is inflated and anchored within a blood vessel. When the balloon catheter is anchored within an intraluminal passage with the work element facing a target lesion on a blood vessel wall, the work element may be lifted such as to press against the target lesion (or a region thereon), thereby compressing and possibly smoothing lesion material on the blood vessel wall. Advantageously, the balloon surface protects the blood vessel walls from direct contact with the work element, thereby decreasing the risk of damage to tissue. The amount of force exerted by the work element may be controllably varied according to characteristics of the lesion, including the stiffness thereof and the shape of the surface thereof. The selective application of force has the advantage of allowing for the resulting lesion material pattern to be smooth rather than wavelike, and, as a further advantage, may eliminate the need for a stent.

Thus, according to an aspect of the present invention, there is provided a catheter system. The catheter system includes:
A guidewire including a proximal segment, a distal segment, and at least one work element. The work element is elongated and extends from a distal end of the proximal segment to a proximal end of the distal segment.
A tube, being flexible and mounted on the guidewire such that the proximal segment longitudinally extends therethrough.
A balloon, being inflatable and enveloping the at least one work element. The balloon is connected at a balloon proximal end to the tube at a distal end thereof.

The guidewire is controllably switchable between a guiding configuration, for maneuvering the catheter system through intraluminal passages, and a work configuration, in which the at least one work element radially projects relative to a longitudinal axis of the balloon and pushes against a surface of the balloon when the balloon is inflated with fluid and anchored in an intraluminal passage. The at least one work element is non-expandable.

According to some embodiments of the catheter system, the at least one work element includes a lesion-smoothing member. The lesion-smoothing member is a rigid or resiliently flexible surface or a rigid or resiliently flexible wire.

According to some embodiments of the catheter system, in the guiding configuration the at least one work element substantially does not project relative to the longitudinal axis.

According to some embodiments of the catheter system, the guidewire is continuously switchable between the guiding configuration and the work configuration, thereby allowing to control the amount of projection of the at least one work element and the amount of force exerted by the at least one work element against the balloon surface when the balloon is inflated with a fluid and anchored in an intraluminal passage.

According to some embodiments of the catheter system, the at least one work element is further configured to allow for controllable rotation about the longitudinal axis, such as to allow the lesion-smoothing member to slide along the balloon surface and simultaneously push there against when the guidewire is in the work configuration and the balloon is inflated with a fluid and anchored in an intraluminal passage; and/or

the at least one work element is further configured to allow for reciprocal motion, such as to allow the lesion-smoothing member to substantially proximally and distally slide along the balloon surface and simultaneously push there against when the guidewire is in the work configuration and the balloon is inflated with a fluid and anchored in an intraluminal passage.

According to some embodiments of the catheter system, the at least one work element is configured to allow for controllable rotation about the longitudinal axis. The controllable rotation is effected by rotating the proximal segment.

According to some embodiments of the catheter system, the mounting of the balloon distal end on the distal segment is such as to prevent any proximal and distal motion of the balloon distal end relative to the distal segment.

According to some embodiments of the catheter system, the catheter system is further configured to allow switching from the guiding configuration to the work configuration by distally pushing the proximal segment when the balloon is anchored.

According to some embodiments of the catheter system, the distal segment includes an exposed segment and a non-exposed segment, located outside of the balloon and within the balloon, respectively. The exposed segment includes a pliable tip at a distal end thereof.

According to some embodiments of the catheter system, the tube distal end includes a first bearing mounted thereon and connected to the balloon proximal end. The proximal segment passes through the first bearing. The distal segment includes a second bearing mounted thereon and connected to the balloon distal end. The first bearing and the second bearing are configured to allow rotating the guidewire without rotating the tube and the balloon. The first bearing is further configured to allow proximal and distal motion therethrough of the proximal segment.

According to some embodiments of the catheter system, the proximal segment includes a first disc, mounted perpendicularly thereto and distally relative to the first bearing such as to be positioned adjacent thereto when the guidewire is in the guiding configuration. The distal segment includes a second disc and a third disc, mounted perpendicularly thereto and proximally and distally relative to the second bearing, respectively, such as to be positioned adjacent thereto.

According to some embodiments of the catheter system, the exposed segment and the non-exposed segment are mechanically associated via a ratchet within the second bearing. The ratchet is configured to allow for (i) joint rotation of the exposed segment together with the non-exposed segment, the at least one work element, and the proximal segment when the proximal segment is rotated in one sense, and (ii) rotation only of the non-exposed segment, the at least one work element, and the proximal segment when the proximal segment is rotated in the other sense.

According to some embodiments of the catheter system, the lesion-smoothing member is substantially convex.

According to some embodiments of the catheter system, the lesion-smoothing member is a convex surface. The at least one work element further includes a first arm, mechanically associating the convex surface on a proximal end thereof with the distal end of the proximal segment, and a second arm, mechanically associating the convex surface on a distal end thereof with the proximal end of the distal segment.

According to some embodiments of the catheter system, the catheter system further includes a first joining region connecting the proximal segment distal end to the first arm, a second joining region connecting the first arm to the convex surface proximal end, a third joining region connecting the convex surface distal end to the second arm, and a fourth joining region connecting the second arm to the distal segment proximal end. The joining regions include hinges, or the joining regions are more flexible than the proximal segment, the first arm, the convex surface, the second arm, and the distal segment, such as to allow the convex surface to be radially lifted when the proximal segment is pushed in the distal direction when the balloon is inflated with a fluid and anchored within an intraluminal passage.

According to some embodiments of the catheter system, each of the joining regions is notched.

According to some embodiments of the catheter system, the proximal segment, the work element, and the non-exposed segment are made of a single wire, which is substantially straight in the guiding configuration. The work element further includes a first arm and a second arm. A first joining region connects the proximal segment distal end to the first arm. A second joining region connects the first arm to a proximal end of the lesion-smoothing member. A third joining region connects a distal end of the lesion-smoothing member to the second arm. A fourth joining region connects the second arm to the distal segment proximal end. The joining regions are notched, with the notches of the first and fourth joining regions being located on the guidewire oppositely (relative to the longitudinal axis) to the second and third joining regions, such as to allow the lesion-smoothing member to be radially lifted when the proximal segment is pushed in the distal direction when the balloon is inflated with a fluid and anchored within an intraluminal passage.

According to some embodiments of the catheter system, the tube is fluidly connected to the balloon, and the tube proximal end is configured to be coupled to a fluid source or vacuum.

According to some embodiments of the catheter system, the catheter system further includes a duct fluidly connected at a distal end thereof to the balloon and configured to be coupled, at a proximal end thereof, to a fluid source or vacuum. The duct is longitudinally joined at least at a distal portion thereof to the tube.

According to some embodiments of the catheter system, the catheter system is further configured to allow for the controllable rotation to be effected by a motor and/or manually.

According to some embodiments of the catheter system, the intraluminal passage is a blood vessel.

According to some embodiments of the catheter system, the blood vessel is a coronary artery or vein.

According to some embodiments of the catheter system, the catheter system includes a plurality of the work element. The plurality of work elements are symmetrically disposed about the longitudinal axis of the balloon.

According to some embodiments of the catheter system, the convex surface is substantially flat.

According to some embodiments of the catheter system, when inflated, the balloon is shaped substantially as an ellipsoid or a cigar.

According to some embodiments of the catheter system, the proximal segment and the distal segment are further connected by a mechanical spring extending along the longitudinal axis.

According to some embodiments of the catheter system, the balloon is a drug-eluting balloon.

According to some embodiments of the catheter system, the work element includes radiographic markers.

According to a further aspect of the present invention, there is provided a method for treating blockage in intraluminal passages. The method includes the steps of:
Introducing a catheter system, according to any one of the embodiments of the catheter system specified above, into an intraluminal passage in a body of a subject, when the catheter system is in the guiding configuration, and maneuvering the catheter system until the balloon reaches a location of a target lesion in the intraluminal passage, with the work element facing the target lesion.
Inflating the balloon with a fluid until the balloon pushes against inner walls of the intraluminal passage, thereby anchoring the balloon.
Switching the catheter system into the work configuration, thereby pushing the work element against the target lesion and compressing the target lesion.

Certain embodiments of the present invention may include some, all, or none of the above advantages. Further advantages may be readily apparent to those skilled in the art from the figures, descriptions, and claims included herein. Aspects and embodiments of the invention are further described in the specification hereinbelow and in the appended claims.

Unless otherwise defined, all technical and scientific terms used herein have the same meaning as commonly understood by one of ordinary skill in the art to which this invention pertains. In case of conflict, the patent specification, including definitions, governs. As used herein, the indefinite articles “a” and “an” mean “at least one” or “one or more” unless the context clearly dictates otherwise.

### BRIEF DESCRIPTION OF THE FIGURES

Some embodiments of the invention are described herein with reference to the accompanying figures. The description, together with the figures, makes apparent to a person having ordinary skill in the art how some embodiments may be practiced. The figures are for the purpose of illustrative description and no attempt is made to show structural details of an embodiment in more detail than is necessary for a fundamental understanding of the invention. For the sake of clarity, some objects depicted in the figures are not to scale.

In the Figures:

FIG. 1a schematically depicts a side-view of a catheter system in a work configuration, according to some embodiments;

FIG. 1b schematically depicts a side-view of the catheter system of FIG. 1a in a guiding configuration, according to some embodiments;

FIG. 1c schematically depicts a side-view of a catheter body of the catheter system of FIG. 1a, according to some embodiments;

FIG. 1d schematically depicts a side-view of a guidewire of the catheter system of FIG. 1a, according to some embodiments;

FIG. 2a schematically a side-view of a work element of the catheter system of FIG. 1a when the catheter system is in the guiding configuration, according to some embodiments;

FIG. 2b schematically a side-view of the work element of the catheter system of FIG. 1a when the catheter system is in the work configuration, according to some embodiments;

FIG. 2c schematically depicts an upper-view of the work element of the catheter system of FIG. 1a when the catheter system is in the work configuration, according to some embodiments;

FIGS. 3a-3e schematically depict compression of a target lesion in a blood vessel, using the catheter system of FIG. 1a, according to some embodiments;

FIG. 4a schematically depicts a side-view of a catheter system—having a work element including a curved wire—in a guiding configuration, according to some embodiments;

FIG. 4b schematically depicts a side-view of the catheter system of FIG. 4a in a work configuration, according to some embodiments;

FIG. 5a schematically depicts a side-view of a catheter system—having a work element including a curved wire and a mechanical spring—in a guiding configuration, according to some embodiments;

FIG. 5b schematically depicts a side-view of the catheter system of FIG. 5a in a work configuration, according to some embodiments;

FIG. 6a schematically depicts a side-view of a portion of a notched guidewire in a guiding configuration, according to some embodiments;

FIG. 6b schematically depicts a side-view of the portion of the notched guidewire of FIG. 6a in a work configuration, according to some embodiments;

FIG. 7a schematically depicts a side-view of a catheter system having a pair of work elements in a guiding configuration, according to some embodiments; and

FIG. 7b schematically depicts a side-view of the catheter system of FIG. 7a in a work configuration, according to some embodiments.

### DETAILED DESCRIPTION OF SOME EMBODIMENTS

The principles, uses and implementations of the teachings herein may be better understood with reference to the accompanying description and figures. Upon perusal of the description and figures present herein, one skilled in the art will be able to implement the teachings herein without undue effort or experimentation. In the figures, like reference numerals refer to like parts throughout.

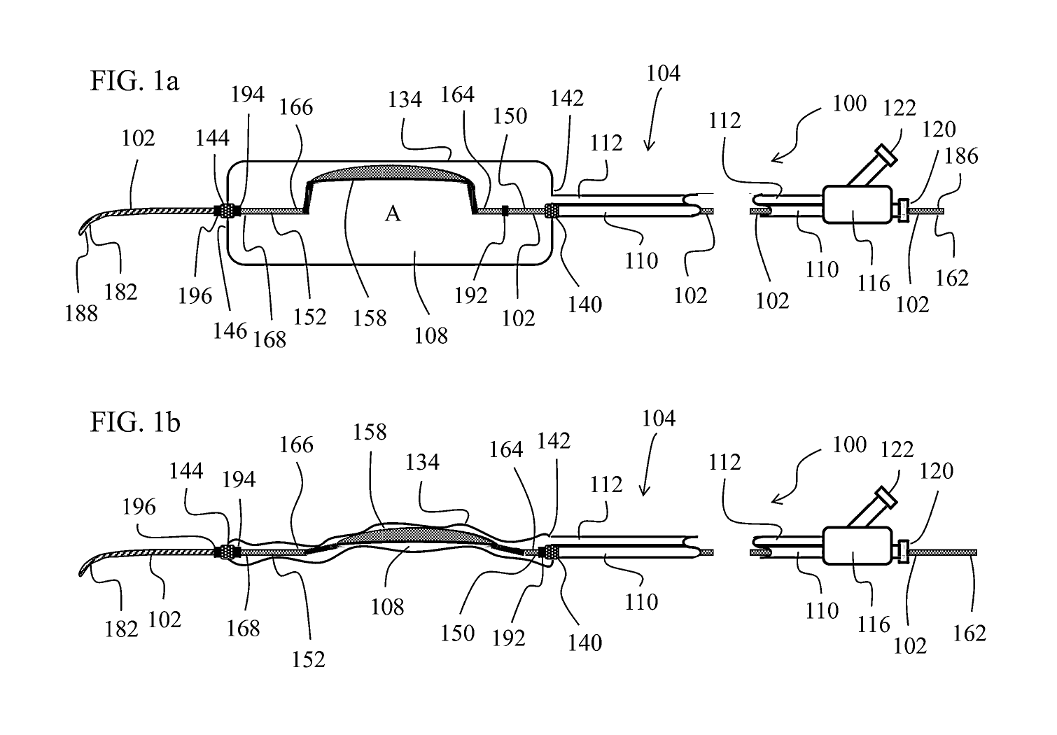

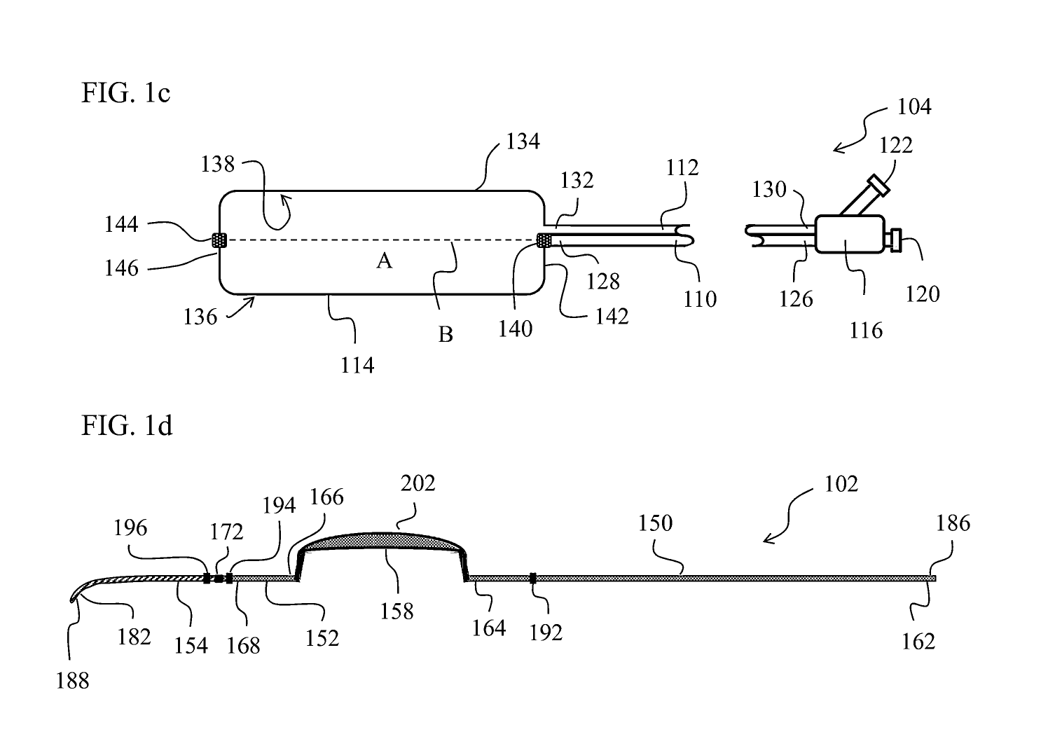

A first exemplary embodiment of a catheter system 100, as described herein, is schematically depicted in FIGS. 1a-1d. Making reference to FIGS. 1a and 1b, catheter system 100 includes a guidewire 102 and a catheter body 104, including an inflatable balloon 108. In FIG. 1a and FIG. 1b guidewire 102 is shown in a work configuration and in a guiding configuration, respectively, and balloon 108 is shown inflated and deflated, respectively. As used herein, catheter system 100 will be said to be in the work configuration when guidewire 102 is in the work configuration, and will be said to be in the guiding configuration when guidewire 102 is in the guiding configuration. The statements “the guidewire is in the guiding configuration” and “the work element is in the guiding configuration” may be used interchangeably. The statements “the guidewire is in the work configuration” and “the work element is in the work configuration” may be used interchangeably.

Making reference also to FIG. 1c, catheter body 104 further includes a flexible tube 110, a flexible duct 112, and a hub 116. Hub 116 includes a guidewire port 120 and an inflation port 122. Tube 110 extends from a tube proximal end 126 to a tube distal end 128. Duct 112 extends from a duct proximal end 130 to a duct distal end 132. Balloon 108 defines a volume A therein (i.e. the volume within balloon 108). Balloon 108 includes a membrane 134 having an external surface 136 and an internal surface 138. Balloon 108 is connected to a first bearing 140 at a balloon proximal end 142 and to a second bearing 144 at a balloon distal end 146, and longitudinally extends along a longitudinal axis B (about which, according to some embodiments, balloon 108 exhibits cylindrical symmetry). First bearing 140 and second bearing 144 are mounted on guidewire 102, as described hereinbelow.

Hub 116 is mounted on the proximal ends of tube 110 and duct 112, i.e. tube proximal end 126 and duct proximal end 130, respectively. Guidewire port 120 is connected to tube 110 by a passageway (not shown) inside hub 116. Inflation port 122 is fluidly associated with duct 112. Tube 110 is connected to first bearing 140 at tube distal end 128. Duct 112 is longitudinally joined to tube 110, and is fluidly connected to balloon 108.

Balloon 108 has a generally cylindrical shape with curved or rounded edges, reminiscent of a cigar, when inflated and no external forces are acting thereon. According to some embodiments, balloon 108 may be shaped as an ellipsoid or a torpedo when inflated and no external forces are acting thereon. According to some embodiments, membrane 134 may be transparent, as depicted in the Figures.

Making reference to FIG. 1d, as well as to FIGS. 1a and 1b, guidewire 102 includes a first segment 150, a second segment 152, a third segment 154, and a work element 158. First segment 150 extends from a first segment proximal end 162, located outside of catheter body 104 (i.e. outside of guidewire port 120), via hub 116, tube 110, and balloon 108, to a first segment distal end 164, whereon first segment 150 connects to work element 158. Second segment 152 is disposed within balloon 108 and extends from a second segment proximal end 166, whereon second segment 152 connects to work element 158, to a second segment distal end 168, whereon second segment 152 connects to third segment 154 via a ratchet 172 (located inside second bearing 144 and therefore not visible in FIGS. 1a and 1b), as elaborated on hereinbelow. Third segment 154 extends in the distal direction from balloon distal end 146 and includes a pliable curved tip 182. Guidewire 102 extends from a guidewire proximal end 186, located at first segment proximal end 162, to a guidewire distal end 188, located at the distal end of curved tip 182.

According to some embodiments, catheter body 104 does not include duct 112. In such embodiments, tube 110 may be fluidly associated with inflation port 122, and, via first bearing 140, which is configured to allow passage of the fluid therethrough, with balloon 108. According to some embodiments, tube 110 and duct 112 are both housed within a single flexible tube (not shown).

Work element 158 is disposed within balloon 108. In FIGS. 1a and 1d, guidewire 102 is shown in the work configuration, whereas in FIG. 1b guidewire 102 is shown in the guiding configuration. The guiding configuration may be used when guiding guidewire 102 and catheter body 104 inside intraluminal passages, and the work configuration may be used when using work element 158 to release stricture in an intraluminal passage, as described hereinbelow. As used herein, the work configuration refers to any of a number of configurations wherein work element 158 radially projects relative to longitudinal axis B (that is to say, work element 158 is lifted relative to longitudinal axis B). According to some embodiments, in the guiding configuration, work element 158 also radially projects relative to longitudinal axis B, the radial projection being smaller than in the work configuration (as depicted, for example, in of FIGS. 1a and 1b).

First bearing 140 is mounted on first segment 150. Second bearing 144 is jointly mounted on second segment 152 and third segment 154. First segment 150 includes a first disc 192, fixedly mounted thereon, inside balloon 108. Second segment 152 includes a second disc 194, fixedly mounted thereon, inside balloon 108, and adjacent to second bearing 144. Third segment 154 includes a third disc 196, fixedly mounted thereon, and adjacent to second bearing 144. First bearing 140 is mechanically barred from distal movement along first segment 150 beyond first disc 192. Second bearing 144 is mechanically barred from both proximal and distal movement relative to second segment 152 (and third segment 154) by second disc 194 and third disc 196, respectively.

As used herein, “distal direction” may refer to the direction along guidewire 102 pointing toward guidewire distal end 188. “Proximal direction” may refer to the direction along guidewire 102 pointing toward guidewire proximal end 186. “Distal movement” may refer to a movement along guidewire 102, or of guidewire 102, in the distal direction. “Proximal movement” may refer to a movement along guidewire 102, or of guidewire 102, in the proximal direction.

First bearing 140 and second bearing 144 are configured to allow guidewire 102 rotation—and, in particular, work element 158 rotation within volume A—independently of catheter body 104. That is to say, guidewire 102 may be rotated without catheter body 104 being simultaneously rotated. Ratchet 172 allows third segment 154 to jointly rotate with second segment 152 (and work element 158 and first segment 150), when second segment 152 is rotating in one sense (e.g. anti-clockwise), but bars third segment 154 from any rotation, when second segment 152 is rotating in the opposite sense (e.g. clockwise).

According to some embodiments, guidewire 102 does not include ratchet 172, and second segment 152 (and work element 158 and first segment 150) cannot be rotated independently of third segment 154. Further, in some such embodiments, second segment 152 and third segment 154 are integrally formed of a single piece of wire.

First bearing 140 and second bearing 144 are further configured such as to substantially prevent any passage of fluid therethrough, and, in particular, to substantially prevent escape therethrough of fluid from within balloon 108. According to some embodiments, first bearing 140 and second bearing 144 may each include O-rings (not shown) mounted on annular grooves therein (not shown), with guidewire 102 passing therethrough, thereby allowing for no passage of fluid through first bearing 140 and through second bearing 144, respectively. Except for being fluidly connected to duct 112, balloon 108 is fluidly, or substantially fluidly, sealed.

According to some embodiments, the “rated burst pressure” (RBP) of a catheter balloon is the maximum pressure at which 99.9% of substantially identical copies of the catheter balloon do not burst with a confidence level of 95%. According to some embodiments, a catheter balloon is “fully inflated” when the pressure therein equals the RBP. A “lateral cross-section” of a catheter balloon, which when inflated is substantially symmetric under rotations about a symmetry axis thereof (e.g. a cylindrical or cigar shaped catheter balloon), refers to a cross-section normal (i.e. perpendicular) to the symmetry axis, e.g. a cross-section of balloon 108 perpendicular to longitudinal axis B. According to some embodiments, the “nominal diameter” of a catheter balloon refers to the diameter of the highest-diameter lateral cross-section of the catheter balloon at “nominal pressure”. According to some embodiments, the “nominal pressure” may be 40% of RBP, or 50% of RBP, or even 60% of RBP. According to some embodiments, the “compliance” of a catheter balloon is defined as the change in the catheter balloon's diameter as a function of the pressure therein. The higher the compliance of a catheter balloon, the more deformable the catheter balloon's shape under application of external forces, when inflated. According to some embodiments, a catheter balloon is said to be “non-compliant” when the diameter of the catheter balloon remains unchanged at pressures above the nominal pressure (but lower than RBP). According to some embodiments, a catheter balloon is said to be “semi-compliant” when the diameter of the catheter balloon may change by up to 10% at pressures above the nominal pressure (but lower than RBP).

As used herein, according to some embodiments, a catheter balloon may be said to be inflated when a volume thereof is at least 67% of a volume thereof at RBP. According to some embodiments, a catheter balloon may be said to be deflated when a volume thereof is no more than 33% of the volume thereof at RBP.

According to some embodiments, the length of balloon 108, i.e. the distance between first bearing 140 and second bearing 144, may range from 1 cm (centimeter) to 6 cm, or from 2 cm to 5 cm, or even from 2 cm to 3 cm. According to some embodiments, the diameter of balloon 108 may range from 0.2 cm to 1 cm, or from 0.3 cm to 0.8 cm. According to some embodiments, balloon 108 is semi-compliant with RBP equaling 16 atm (atmosphere) or 20 atm, or even 24 atm, and the nominal pressure equaling 50% of RBP or even 60% of RBP. According to some embodiments, balloon 108 is non-compliant with RBP equaling 8 atm or 12 atm or even 14 atm, and the nominal pressure equaling 50% of RBP or even 60% of RBP.

Balloon 108 may be made of nylon, PVC (polyvinyl chloride), PET (polyethylene terephthalate), PP (polypropylene), latex, silicon, or the like, as known in the art.

According to some embodiments, the length of guidewire 102 may range from 1 m (meter) to 4 m. According to some embodiments, the length of guidewire 102 may range from 1.5 m to 2.5 m. According to some embodiments, the diameters of first segment 150 and second segment 152 may range from 0.02 cm to 0.1 cm. According to some embodiments, the diameters of first segment 150 and second segment 152 may range from 0.03 cm to 0.05 cm, or even from 0.033 cm to 0.037 cm. According to some embodiments, the length of third segment 154 may range from 2 cm to 5 cm, or from 3 cm to 4 cm. According to some embodiments, the diameter of third segment 154 may range from 0.02 to 0.1 cm or from 0.03 cm to 0.05 cm, or even from 0.033 cm to 0.037 cm. According to some embodiments, third segment 154 diameter may taper in the distal direction.

First segment 150, second segment 152, and third segment 154 are sufficiently stiff to allow guidewire 102 to be advanced through intraluminal passages in a subject's body, e.g. blood vessels, and at the same time are sufficiently flexible to allow guidewire 102 to be guided through bends therein the blood vessels. Depending on a target intraluminal passage, first segment 150, second segment 152, and third segment 154, may each have a different stiffness. First segment 150 and second segment 152 may be made of a flexible metal, as known in the art. Third segment 154 may be made of the same flexible metal, or another flexible metal. According to some embodiments, curved tip 182 includes a spring coil, as known in the art. According to some embodiments, each of first segment 150, second segment 152, and third segment 154 are made of a different material (e.g. a different flexible metal), respectively.

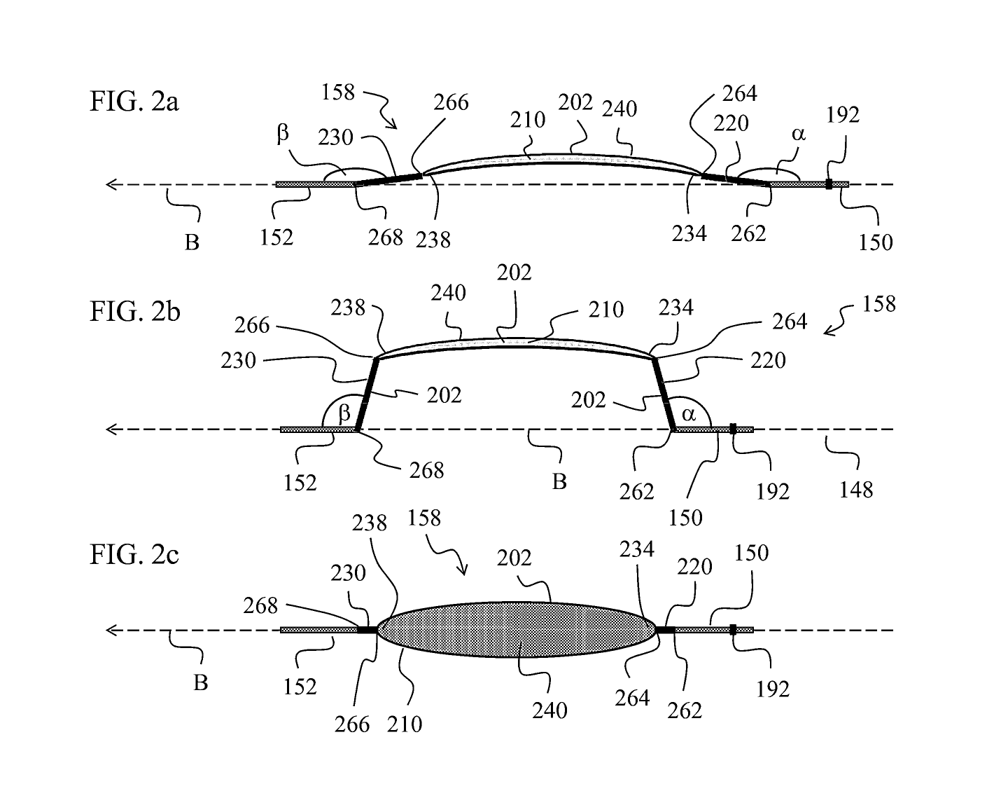

FIGS. 2a-2c schematically depict work element 158. FIG. 2a schematically depicts a side-view of work element 158 when guidewire 102 is in the guiding configuration. Work element 158 includes a lesion-smoothing member 202 having a convex surface 210. Work element 158 further includes a first arm 220, and a second arm 230. Convex surface 210 is elongated and includes a surface proximal end 234 and a surface distal end 238. Convex surface 210 further includes an outer face 240 and an inner face (not numbered). First arm 220 connects via a first joining region 262 to first segment 150 and via a second joining region 264 to surface proximal end 234. Second arm 230 connects via a third joining region 266 to surface distal end 238 and via a fourth joining region 268 to second segment 152. Joining regions 262, 264, 266, and 268 form longitudinally narrow regions (i.e. regions of small longitudinal extent) of reduced stiffness along guidewire 102, as elaborated on hereinbelow. According to some embodiments, outer face 240 is smooth. According to some alternative embodiments, outer face 240 is flat or substantially flat (as used herein, a flat surface may also be referred to as convex).

According to some embodiments, in the guiding configuration convex surface 210 projects radially from longitudinal axis B, with first arm 220 forming an angle α with first segment 150, and with second arm 230 forming an angle β with second segment 152. α and β may be substantially equal, assuming a value of 165°, 170°, 175°, or even 180°. When no external forces are exerted on guidewire 102, work element 158 is in the guiding configuration.

FIG. 2b schematically depicts a side-view of work element 158 when guidewire 102 is in the work configuration. In the work configuration, work element 158 radially projects from longitudinal axis B. In embodiments wherein work element 158 radially projects from longitudinal axis B also in the guiding configuration (e.g. when α and β are smaller than 180° in the guiding configuration, and/or wherein convex surface 210 is not flat or substantially flat), the radial projection of work element 158 is greater in the work configuration than in the guiding configuration. According to some embodiments, (in the work configuration) the values of α and β may controllably be set anywhere in a range from 165° to 90°, thereby controlling the amount of radial projection or lifting of convex surface 210 relative to longitudinal axis B.

FIG. 2c schematically depicts an upper-view of work element 158 when guidewire 102 is in the work configuration (i.e. in FIG. 2c the orientation of guidewire 102 and of work element 158 is rotated by 90° about longitudinal axis B relative to the orientation thereof in FIG. 2b). Outer face 240 is fully visible. When work element 158 is rotated by 360° about longitudinal axis B (that is to say, when work element 158 effects a full revolution about longitudinal axis B), due to convex surface 210 convexity, convex surface 210 rotation may define a substantially ellipsoid-like surface.

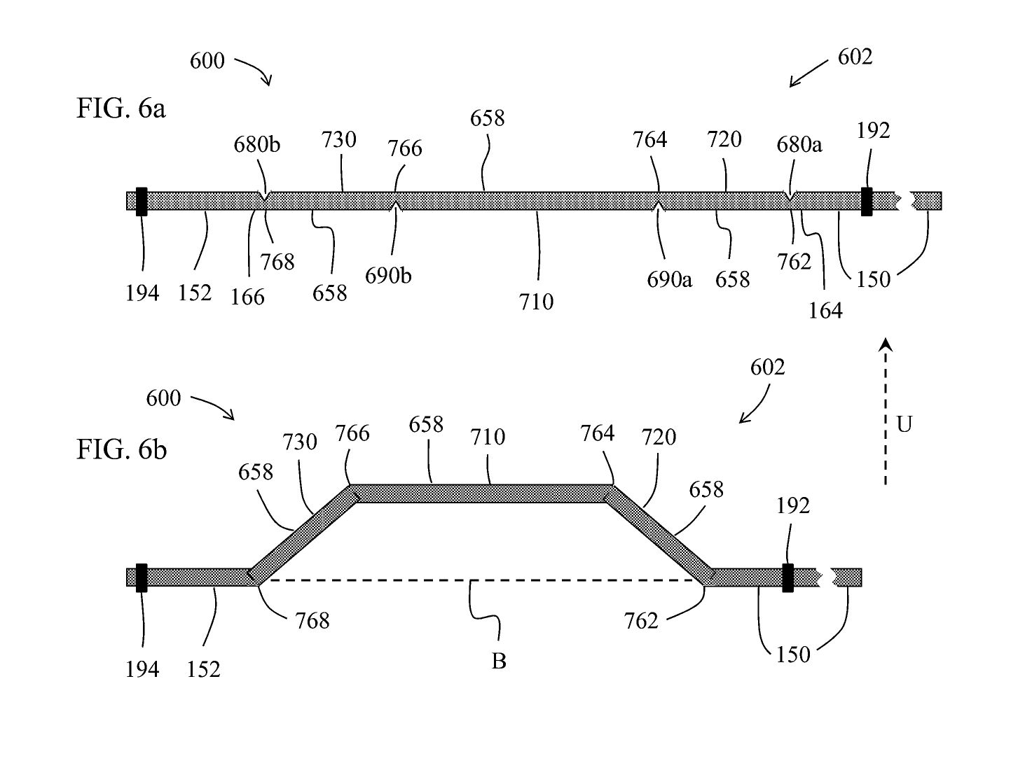

According to some embodiments, convex surface 210 is rigid. According to some embodiments, convex surface 210 is resiliently flexible. According to some embodiments, first arm 220 and second arm 230 are rigid. Joining regions 262, 264, 266, and 268 are each more flexible than first segment 150, second segment 152, first arm 220, second arm 230, and convex surface 210. According to some embodiments, joining regions 262, 264, 266, and 268 may be made of a more flexible material than first segment 150, second segment 152, first arm 220, and second arm 230 (and convex surface 210). According to some embodiments, joining regions 262, 264, 266, and 268 are narrower (thinner) than first segment 150, second segment 152, first arm 220, and second arm 230. According to some embodiments, joining regions 262, 264, 266, and 268 are notched, essentially as described in the description of FIGS. 6a-6b.

According to some embodiments, convex surface 210 is substantially non-expandable (e.g. in contrast to a balloon or a rubber band). As used herein, a solid object or element may be said to be “non-expandable” even while being resiliently flexible. A typical plastic clipboard (e.g. for writing) provides an example of a non-expandable yet resiliently flexible solid object. Another example is provided by a rubber hose (e.g. for hand-watering a garden).

To switch from the guiding configuration to the work configuration, a force in the distal direction may be applied on first arm 220 via first joining region 262. When balloon 108 is anchored within an intraluminal passage, due to second segment distal end 168 position being fixed and due to joining regions 262, 264, 266, and 268 having a smaller stiffness than first segment 150, second segment 152, first arm 220, second arm 230, and convex surface 210, joining regions 262, 264, 266, and 268 may bend such as to decrease α and β, thereby radially lifting convex surface 210 and simultaneously distally shifting convex surface 210. More specifically, according to some embodiments, the distal force applied on first arm 220 results in torques being applied respectively on first arm 220 and on second arm 230 (on second arm 230 via surface distal end 238), thereby lifting convex surface 210. According to some embodiments, values of α and β close to (but smaller than) 180° in the guiding configuration may help in preventing scenarios wherein either one of α and β, or both, increases under the applied force and convex surface 210 is not radially lifted. By varying the distal force exerted, the values of α and β may be controllably varied, and thereby the degree or amount of lifting of convex surface 210.

It is noted that by alternatingly simultaneously increasing α and β, and simultaneously decreasing α and β, a back-and-forth motion of work element 158 is effected. When the increase and decrease in α and β are sufficiently small, the back-and-forth motion will be mainly in parallel to longitudinal axis B, as elaborated on hereinbelow.

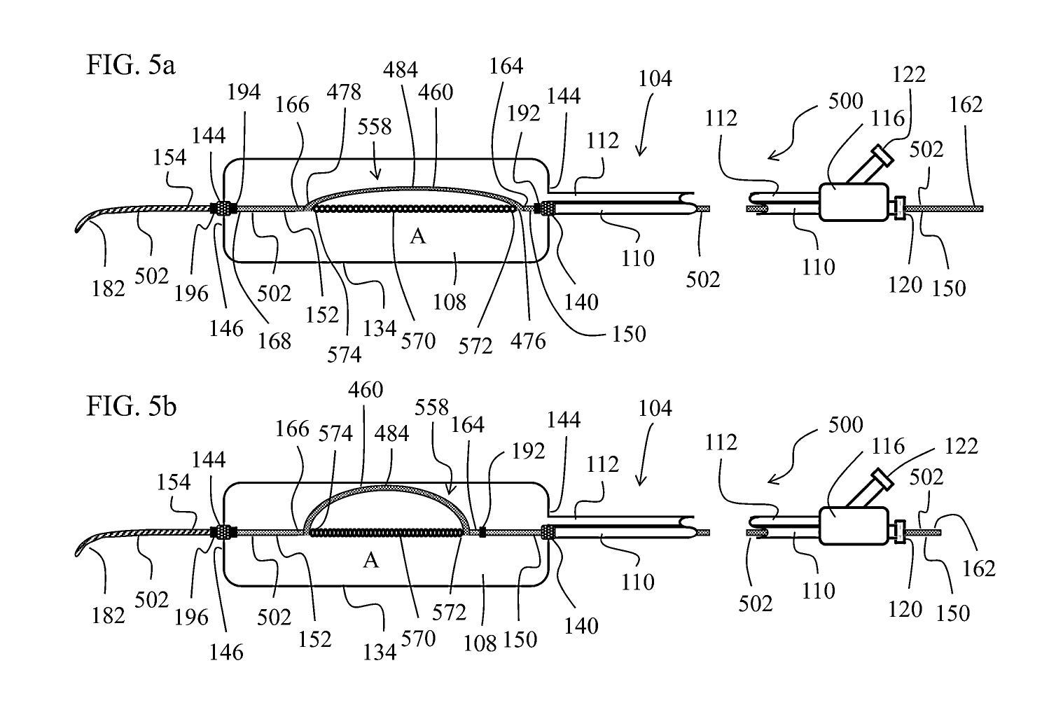

It is further noted that as convex surface 210 is lifted, α and β may decrease at different rates. For example, as first segment 150 starts being pushed, a may decrease (e.g. from 180° or) 175° at a faster rate than β, resulting in surface proximal end 234, at least initially, projecting further from longitudinal axis B than surface distal end 238. According to some embodiments, first arm 220 and second arm 230 do not have the same length. According to some embodiments, first arm 220 is shorter than second arm 230. According to some embodiments, joining regions 262 and 268 are further connected by a mechanical spring (similar to the mechanical in FIGS. 5a-5b), which extends along longitudinal axis B. According to some embodiments, the provision of extra stiffness by the mechanical may help in balancing the rates of increase of α and β.

To switch from the work configuration to the guiding configuration, the application of the distal force on first arm 220 is stopped (ceased). If necessary, a force in the proximal direction may be applied on first arm 220 via first joining region 262, thereby proximally pulling work element 158. According to some embodiments, when no forces are acting on work element 158, guidewire 102 is in the guiding configuration.

According to some embodiments, first segment 150 may be mounted through a chuck (not shown), as used in a drill, proximally located relative to hub 116. The chuck may be housed within a chuck housing (not shown). The chuck housing may be stationary, for example, being secured to an operating table, and thereby configured to prevent displacement of the chuck, particularly, proximal and distal displacement of the chuck, while simultaneously allowing the chuck to be rotated.

The chuck admits a locked configuration and an unlocked configuration. In the locked configuration, the chuck grips guidewire 102, thereby preventing guidewire 102 from distal and proximal motion. When the chuck is rotated, the chuck's grip on guidewire 102 causes guidewire 102 to be jointly rotated with the chuck. According to some embodiments, the chuck may be controllably rotated using an electric motor. According to some embodiments, the chuck may be controllably rotated manually. In the unlocked configuration, the chuck does not grip guidewire 102, and guidewire 102 distal and proximal motion are allowed. Rotation of the chuck does not induce guidewire 102 rotation, and guidewire 102 may be rotated independently of the chuck.

When guidewire 102 is in the guiding configuration, the chuck is unlocked. After guidewire 102 has been switched to the work configuration, the chuck may be locked, thereby preventing first segment 150 proximal movement, and, in particular, guidewire 102 return to the guiding configuration.

Other embodiments, allowing to controllably switch between the work configuration and the guiding configuration, are contemplated. According to some embodiments, joining regions 262, 264, 266, and 268 include one-way hinges (not shown). According to some embodiments, joining regions 262, and 268 include hinges restricting α and β to vary, for example, between 180° and 90°, or between 175° and 90°. Similarly, joining regions 264 and 266 may include hinges which restrict the ranges of the angles (not indicated) between first arm 220 and convex surface 210 and second arm 230 and convex surface 210, respectively.

According to some embodiments, work element 158 includes radiographic markers (not shown). According to some embodiments, some of the markers are located on outer face 240. Using standard imaging techniques known in the art, e.g. fluoroscopy, the markers may help in visualizing work element 158, including the location of convex surface 210 within balloon 108, when balloon 108 is inside an intraluminal passage.

According to some embodiments, e.g. wherein balloon 108 is compliant, in the work configuration, when balloon 108 is fully inflated outside an intraluminal passage, convex surface 210 does not push against internal surface 138, or is not in contact with internal surface 138 (as depicted for example in FIG. 1a). According to some embodiments, e.g. wherein balloon 108 is semi-compliant, in the work configuration, when balloon 108 is fully inflated outside an intraluminal passage, convex surface 210 pushes against internal surface 138, or is in contact with internal surface 138.

As used herein, “lesion” may refer to an abnormality of/in an intraluminal passage resulting in stenosis, including the formation of cholesterol, fat, calcium deposits (e.g. plaque) and/or the like, the formation of blood clots, growth of scar tissue and/or the like within blood vessels. As used herein, “stenosis” may refer to a narrowing of an intraluminal passage, including blood vessels, and may be used interchangeably with stricture and blockage. As used herein, “blockage” may refer to an impediment to fluid flow in an intraluminal passage due to narrowing thereof, and according to some embodiments may be used interchangeably with “partial blockage”. As used herein, “blockage release” in a stenosed region of an intraluminal passage, may refer to a removal or partial removal of the impediment to the fluid flow (e.g. blood flow) in the stenosed region, e.g. in the broadening of the intraluminal passage in the stenosed region.

According to an aspect of some embodiments, there is provided a method for releasing stenosis in a target blood vessel, using catheter system 100 or a catheter system similar thereto (such as catheter systems 400, 500, and 800, disclosed hereinbelow). Throughout all the steps listed below, standard imaging techniques known in the art, e.g. fluoroscopy, may be used to visualize parts of guidewire 102 and catheter body 104 inside blood vessels. The method includes the following steps:

In a step I, balloon 108 is in a deflated state, containing a small amount of fluid (e.g. a saline 5% solution), and guidewire 102 is in the guiding configuration. Third segment 154 is inserted into a main blood vessel in the subject's body (e.g. a femoral artery in a leg of the subject (patient) when the target blood vessel is a coronary artery) and guided onto the target blood vessel.

According to some embodiments, third segment 154 may be advanced in a blood vessel by distally pushing first segment 150 from the outside of catheter body 104, i.e. pushing first segment 150 further into guidewire port 120. First segment 150 distal push is translated, via work element 158 and second segment 152, into third segment 154 distal push. As guidewire 102 is advanced in the blood vessel, second bearing 144, being distally pushed by second disc 194, pulls balloon 108 therewith. Balloon 108 pulls the rest of catheter body 104 (tube 110, duct 112 and hub 116).

According to some embodiments, third segment 154 may be advanced in the blood vessel by distally pushing tube 110 from outside the subject's body. Tube 110 distal push is translated via first bearing 140 into first disc 192 distal push, and, thereby, via first segment 150, work element 158, and second segment 152, into third segment 154 distal push. According to some embodiments, catheter system 100 does not include first disc 192 and third disc 196, and third segment 154 may be advanced by distally pushing first segment 150, as described hereinabove.

Third segment 154 may be guided into an opening of a second blood vessel, diverging from a first blood vessel, wherein curved tip 182 is located, by rotating anti-clockwise first segment 150 from the outside of catheter body 104. First segment 150 anti-clockwise rotation is translated via work element 158 and second segment 152 into third segment 154 anti-clockwise rotation. The rotation is continued until curved tip 182 points towards the opening of the second blood vessel. First segment 150, and/or tube 110, may then be distally pushed, causing third segment 154 to advance into the second blood vessel.

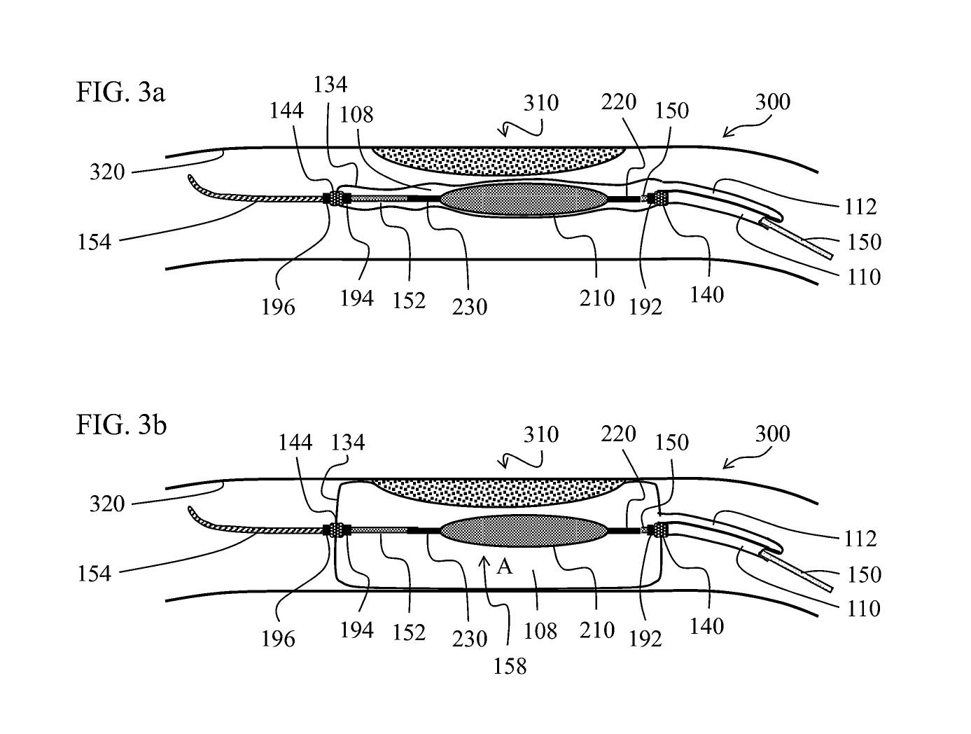

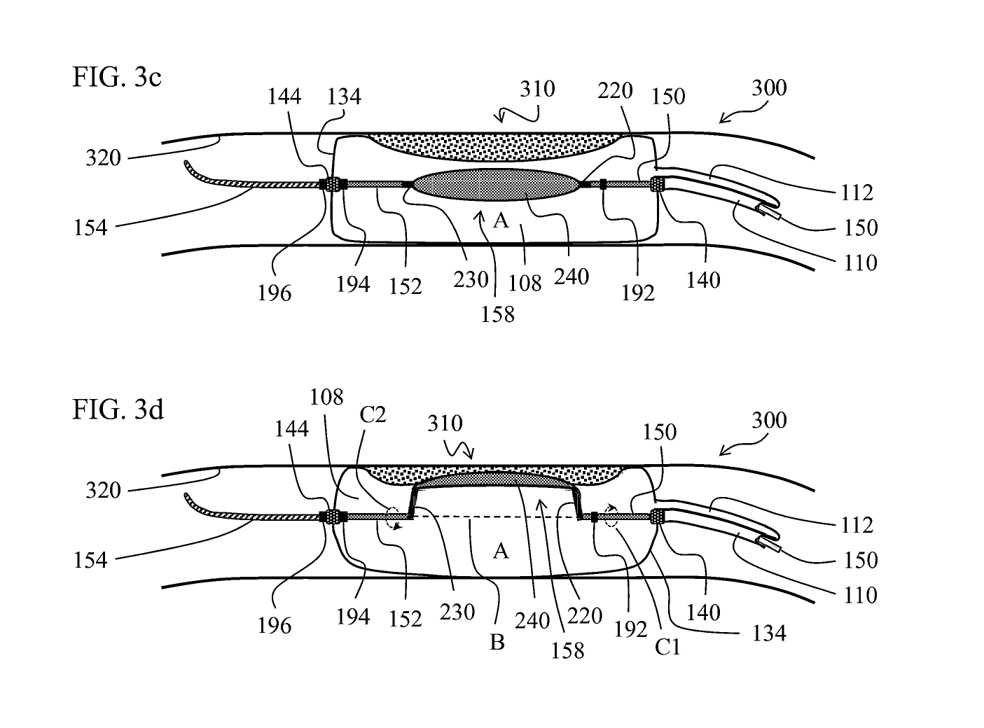

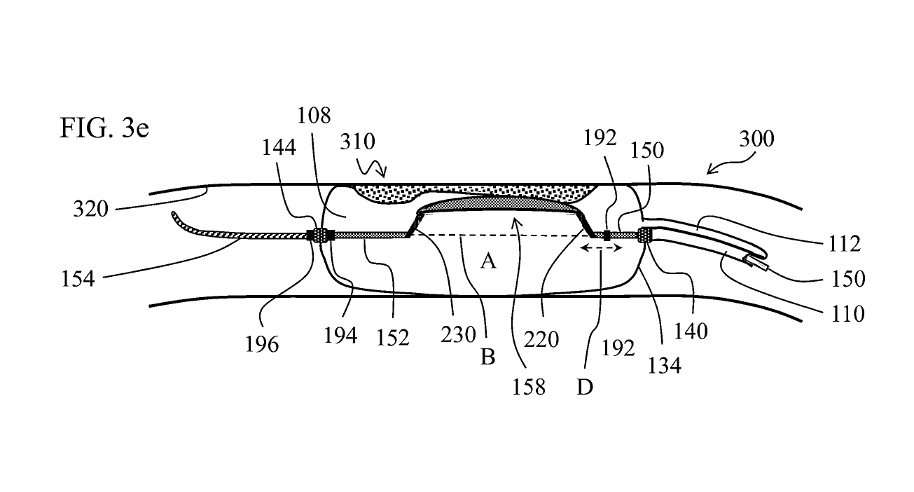

Third segment 154 is guided through a series of diverging blood vessels into the target blood vessel and advanced therein until balloon 108 reaches a stenosed target region in the target blood vessel (as schematically depicted in FIG. 3a). A precise location, whereunto balloon 108 is advanced, may be selected such that convex surface 210 will be adjacent to a target lesion (in the stenosed target region) following the distal shift in convex surface 210 position involved in switching guidewire 102 to the working configuration (in step III below).

In a step II, balloon 108 is inflated by pumping fluid therein via inflation port 122. When a sufficient amount of fluid has been pumped into balloon 108, membrane 134 comes into contact with the surrounding blood vessel walls (as schematically depicted in FIG. 3b). As more fluid is pumped into balloon 108, membrane 134 starts pushing against the surrounding blood vessel walls, thereby anchoring balloon 108 within the blood vessel, and, in particular, preventing distal and proximal movement or dislocation of the balloon, as further elaborated on hereinbelow. Balloon 108 inflation may partially compress the target lesion.

According to some embodiments, balloon 108 may be fully inflated (i.e. up to RBP). According to some embodiments, balloon 108 may inflated to the nominal pressure. According to some embodiments, following inflation, balloon 108 may be partially deflated, such that a pressure within balloon 108 substantially equals 90% of the nominal pressure, 70% of nominal pressure, or even 40% of the nominal pressure. The partial deflation may increase the effective elasticity of balloon 108 and bring it to a desired level. The higher the compliance of balloon 108, the less deflation may be required to achieve the desired level of effective elasticity. According to some embodiments, balloon 108 may be inflated to a pressure as high as 24 atm. According to some embodiments, balloon 108 may be inflated to a pressure as low as 2 atm.

In a step III, guidewire 102 is distally pushed (i.e. into the main blood vessel and therefore further into the target blood vessel) by distally pushing first segment 150 from the outside of catheter body 104, i.e. pushing first segment 150 further into guidewire port 120. First segment 150 distal push is translated via work element 158, second segment 152, and second disc 194 to a distal push on second bearing 144, which in turn distally pulls on balloon 108. Since balloon 108 is inflated, balloon 108 is prevented from movement by surrounding blood vessel walls and lesions thereon, and, in particular, from distal movement (i.e. movement up the target blood vessel), and, consequently, so is second bearing 144. According to some embodiments, due to joining regions 162, 164, 166, and 168 being more flexible than first segment 150, first arm 220, convex surface 210, second arm 230, and second segment 152, as a result of the distal push on first segment 150, torques are applied on first arm 220 and second arm 230, respectively. Convex surface 210 is thereby radially lifted, bringing guidewire 102 into the work configuration, as explained hereinabove in the description of FIGS. 2a-2c (and as schematically depicted in FIG. 3c).

As convex surface 210 is radially lifted, outer face 240 comes into contact with internal surface 138, thereby exerting pressure on membrane 134. The pressure on membrane 134 is translated into a radial (i.e. outward) force acting on an adjacent region of the target lesion.

According to some embodiments, guidewire 102 is mounted through a chuck, such as the chuck described hereinabove, and guidewire 102 may be prevented from returning to the guiding configuration by locking the chuck.

In a step IV, first segment 150 may be clockwise rotated. First segment 150 clockwise rotation is translated into work element 158 and second segment 152 clockwise rotation. Second segment 152 clockwise rotation is not translated into third segment 154 clockwise rotation due to ratchet 172 being configured to prevent joint clockwise rotation of second segment 152 and third segment 154. The clockwise rotation may be manually effected by manually rotating first segment 150 from the outside of catheter body 104, or automatically effected by using an external motor (not shown) to rotate first segment 150.

As work element 158 is clockwise rotated, it slides on, and pushes against, internal surface 138, thereby coming into mechanical contact (that is to say, indirect contact mediated by membrane 134) with adjacent regions of the target lesion, causing a compression of lesion material in the adjacent regions (as schematically depicted in FIG. 3d). The speed of the rotation may be varied, thereby increasing a force exerted by convex surface 210 on the adjacent regions. The rotation may be continued until the target lesion has been sufficiently evenly smoothed on surrounding inner walls of the target blood vessel.

According to some embodiments, step IV may include an anti-clockwise rotation of first segment 150. First segment 150 anti-clockwise rotation is translated into work element 158, second segment 152, and third segment 154 anti-clockwise rotation. A combination of clockwise and anti-clockwise rotations may improve the smoothing of the lesion on the inner walls of the target blood vessel.

When the target blood vessel includes sharp bends near the region of the lesion, rotating third segment 154 may force curved tip 182 into the blood vessel walls (e.g. the walls surrounding the target region). By rotating clockwise first segment 150, third segment 154 rotation is avoided and curved tip 182 is kept away from the blood vessel walls.

According to some embodiments, wherein guidewire 102 is mounted through the chuck, guidewire 102 controllable rotation may be induced by controllably rotating the chuck. According to some embodiments, the chuck may be controllably rotated using a motor. In some such embodiments, the speed of the rotation is controllable. In some such embodiments, the speed of the rotation may be controllably modified during work element 158 rotation.

According to some embodiments, a proximal portion (not numbered in the Figures) of first segment 150 includes a crank mechanism (e.g. a portion of first segment 150 which radially projects relative to the rest of first segment 150, as known in the art), thereby allowing manually rotating first segment 150 and work element 158. According to some embodiments, guidewire 102 may both be mounted through a chuck and include a crank mechanism.

According to some embodiments, step IV may include effecting reciprocating motion (that is to say, back-and-forth proximal and distal motion) of first segment 150. First segment 150 reciprocating motion translates into work element 158 reciprocating motion (as schematically depicted in FIG. 3e). According to some embodiments, the reciprocating motion may involve small displacements of work element 158 along a path (no indicated) which is close to parallel to longitudinal axis B. For example, the small displacements may be such that the radial projection of convex surface 210 does not drop below, for example, 95% of the maximum radial projection thereof, implying that, in some embodiments wherein α and β may be set to 90° (whereat the maximum radial projection is attained), in the reciprocating motion α and β will continuously switch between 90° and about 108°. According to some embodiments, the reciprocating motion may involve larger displacements of work element 158, wherein the radial projection of convex surface 210 does not drop below, for example, 75% of a maximum radial projection thereof, implying that, in some embodiments wherein a and β may be set to 90°, in the reciprocating motion α and β will continuously switch between 90° and about 131°.

The reciprocating motion may be manually effected by manually alternatingly pushing and pulling first segment 150 from the outside of catheter body 104, or automatically effected by using an external motor (not shown) to alternatingly push and pull first segment 150.

According to some embodiments, balloon 108 is a drug-eluting balloon, and may be configured to release a drug, which coats external surface 136, when inflated to the nominal pressure or to a pressure above the nominal pressure, for example, in step II. The drug may also be released from a particular region of membrane 134 even when the pressure within balloon 108 is lower than the nominal pressure, but membrane 134 surface tension in the particular region is high, for example, due to a force exerted on the particular region by convex surface 210 during work element 158 rotation in step IV.

In a step V, work element 158 is switched back to the guiding configuration by controllably releasing first segment 150, for example, by unlocking the chuck in embodiments wherein guidewire 102 is mounted therethrough. Balloon 108 is deflated by pumping out the fluid therein via inflation port 122.

In a step VI, guidewire 102 and catheter body 104 may be pulled out of the subject's body by proximally pulling first segment 150 from the outside of catheter body 104. First segment 150 proximal pull may be translated via first disc 192 and/or third disc 196 via first bearing 140 and/or second bearing 144, respectively, to balloon 108 and catheter body 104 distal pull. If necessary, third segment 154 (anti-clockwise) rotation may be induced by anti-clockwise rotating guidewire 102, as described hereinabove.

According to some embodiments, guidewire 102 and catheter body 104 may be pulled out of the body by proximally pulling tube 110 from outside the subject's body. Tube 110 proximal pull is translated via second bearing 144 and second disc 194 to second segment 152 proximal push at second segment distal end 168, which then translates into a proximal push of first segment 150 and a proximal pull of third segment 154.

According to some embodiments, following step V and prior to step VI, guidewire 102 may be used to guide balloon 108 to another stenosed region and steps II to V may be repeated.

FIGS. 3a-3e schematically depict stenosis release in a blood vessel 300 according to some embodiments of the method described hereinabove. Blood vessel 300 includes a lesion 310 on a vessel inner wall 320.

FIG. 3a schematically depicts parts of catheter system 100 at the end of step I or at the beginning of step II, according to some embodiments. Balloon 108 is still deflated, or mostly deflated, and is positioned adjacent to lesion 310. Guidewire 102 is in the guiding configuration.

FIG. 3b schematically depicts parts of catheter system 100 at the end of step II. Balloon 108 has been inflated. Balloon 108 is pressing against vessel inner wall 320 and lesion 310, and as a result balloon 108 shape is deformed from the cylindrical shape. Guidewire 102 is in the guiding configuration.

FIG. 3c schematically depicts parts of catheter system 100 at the end of step III, according to some embodiments. Balloon 108 is inflated, pressing against vessel inner wall 320 and lesion 310, and as a result is deformed, as explained hereinabove. Guidewire 102 is in the work configuration.

FIG. 3d schematically depicts parts of catheter system 100 in step IV, according to some embodiments. Balloon 108 is inflated, pressing against vessel inner wall 320 and lesion 310, and thereby deformed, as explained hereinabove. Guidewire 102 is in the work configuration and work element 158 is being clockwise rotated. Curled arrows C1 and C2, which curl around first segment 150 and second segment 152, respectively, and point in the clockwise direction, indicate first segment 150 clockwise rotation (and thereby work element 158 clockwise rotation) and second segment 152 clockwise rotation. Work element 158 clockwise rotation results in the smoothing (compression) against vessel inner wall 320 of lesion material in regions of lesion 310, which come into mechanical contact with outer face 240 (due to work element 158 rotation).

FIG. 3e schematically depicts parts of catheter system 100 in step IV, according to some embodiments. Balloon 108 is inflated, pressing against vessel inner wall 320 and lesion 310, and thereby deformed, as explained hereinabove. Guidewire 102 is in the work configuration. First segment 150 is being made to effect reciprocal motion along longitudinal axis B, as indicated by double-headed arrow D, resulting in work element 158 back-and-forth motion.

Work element 158 back-and-forth motion results in the smoothing (compression) against vessel inner wall 320 of lesion material in regions of lesion 310, which come into mechanical contact with outer face 240 (due to work element 158 reciprocating motion).

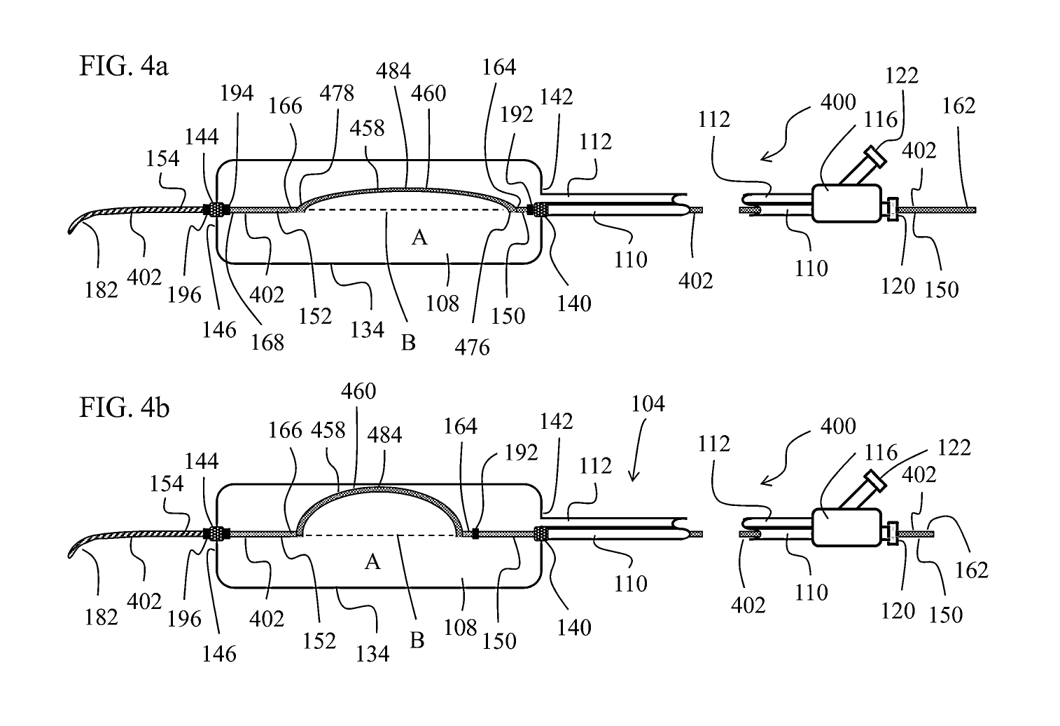

Another exemplary embodiment of a catheter system, as described herein, is schematically depicted in FIGS. 4a-4b. Making reference to FIG. 4a, a side-view of a catheter system 400, including catheter body 104 and a guidewire 402, is shown in a guiding configuration, with balloon 108 inflated. Guidewire 402 includes first segment 150, second segment 152, third segment 154, and a work element 458.

According to this embodiment, work element 458 includes a lesion-smoothing member in the form of a curved wire 460. Curved wire 460 includes a curved wire proximal end 476, which is connected to first segment 150 at first segment distal end 164, and a curved wire distal end 478, which is connected to second segment 152 at second segment proximal end 166. That is, guidewire 402 is essentially similar to guidewire 102 except for including work element 458 in place of work element 158.

Curved wire 460 radially projects from longitudinal axis B, and, according to some embodiments, is convex (as depicted in FIG. 4a). A curved wire top 484 defines a location along curved wire 460, which is more distant from longitudinal axis B than any other location along curved wire 460. According to some embodiments, first segment 150, work element 458, and second segment 152 may be made of a single flexible metallic wire, with work element 458 including a pre-shaped curved or convex portion of the wire. According to some embodiments, curved wire 460 has the shape of an arc. According to some embodiments, curved wire 460 is thicker than first segment 150 and second segment 152.

FIG. 4b schematically depicts a side-view of catheter system 400 in a work configuration. In the work configuration, curved wire 460 radially projects further from longitudinal axis B than in the guiding configuration. That is to say, in the work configuration curved wire 460 is more curved than in the guiding configuration, and curved wire top 484 is situated further from longitudinal axis B than in the guiding configuration. Furthermore, in the work configuration, a position of curved wire 460 inside balloon 108 is distally shifted as compared to the position therein in the guiding configuration (a position of curved wire proximal end 476 is distally shifted, while a position of curved wire distal end 478 remains unchanged).

Guidewire 402 may be brought to the work configuration from the guiding configuration by distally pushing first segment 150, thereby exerting on curved wire 460 a distal force at curved wire proximal end 476. Since second segment distal end 168 is fixed, by varying the distal force, a curvature of curved wire 460 may be controllably varied, thereby varying a height of curved wire top 484. As used herein, the height of curved wire top 484 may refer to the distance from curved wire top 484 to longitudinal axis B. When no external forces are acting on curved wire 460, guidewire 402 is in the guiding configuration.

Catheter system 400 may be used to release stenosis in a target blood vessel in essentially the same way as catheter system 100 (and as described hereinabove). To bring guidewire 402 from the guiding configuration to the work configuration, in a step such as step III (i.e. when catheter system 400 is inserted into a subject's body, such that balloon 108 and work element 458 are in a target blood vessel at a region of stenosis, and balloon 108 is inflated), first segment 150 distal push is translated via work element 458 and second segment 152 to a distal push on second bearing 144, which in turn distally pulls on balloon 108. Since balloon 108 is inflated, balloon 108 is prevented from movement by surrounding blood vessel walls and lesions thereon, and, in particular, from distal movement (i.e. movement up the target blood vessel), and, consequently, so is second bearing 144. Due to curved wire 460 being curved and convex, curved wire 460 may be more readily bent than first segment 150 and second segment 152, and the distal push on first segment 150 results in guidewire 402 switching to the work configuration and the height of curved wire top 484 being increased. According to some embodiments, curved wire 460 is made of a more resiliently flexible material than first segment 150 and second segment 152.

According to some embodiments, catheter system 400 admits only the guiding configuration. In such embodiments, the height of curved wire top 484 may be comparable to the height of same in the work configuration in embodiments, which also admit the work configuration. The above-described method for stenosis release may still be used with step III omitted, the switching back from the work configuration to the guiding configuration in a step, such as step V, omitted, and with the guiding configuration being used also for the compression of the target lesion, i.e. in a step such as step IV.

Making reference again to catheter system 100, according to some embodiments, work element 158 does not include first arm 220 and second arm 230, and convex surface 210 is connected to first segment distal end 164 and second segment proximal end 166 at surface proximal end 234 and surface distal end 238, respectively. In such embodiments, convex surface 210 is resiliently flexible, increasing the convexity thereof, and thereby the radial projection thereof, when a distal force is applied at surface proximal end 234 (and when second segment proximal end 166 is substantially fixed, such as when balloon 108 is anchored), essentially as described above with respect to curved wire 460.

Another exemplary embodiment of a catheter system, as described herein, is schematically depicted in FIGS. 5a-5b. Making reference to FIG. 5a, a side-view of a catheter system 500, including catheter body 104 and a guidewire 502, is shown in a guiding configuration, with balloon 108 inflated. Guidewire 502 includes first segment 150, second segment 152, third segment 154, and a work element 558.

Work element 558 includes a lesion-smoothing member in the form of curved wire 460 and a mechanical spring 570. That is to say, work element 558 is essentially similar to work element 458 except for further including mechanical spring 570, with curved wire top 484 having substantially the same height, as the height thereof in the guiding configuration of catheter system 400. Mechanical spring 570 extends along longitudinal axis B (not visible in FIGS. 5a and 5b due to mechanical spring 570) and is connected at a proximal spring end 572 to curved wire proximal end 476 and at a distal spring end 574 to curved wire distal end 478. In the guiding configuration mechanical spring 570 may be loose.

FIG. 5b schematically depicts a side-view of catheter system 500 in a work configuration. In the work configuration curved wire 460 radially projects further from longitudinal axis B than in the guiding configuration, with curved wire top 484 having substantially the same height as the height thereof in the work configuration of catheter system 400. Mechanical spring 570 is compressed, and prevented from being released, for example, by a chuck and a stationary chuck housing, such as the chuck and the chuck housing described hereinabove in the description of catheter system 100.

Mechanical spring 570 provides extra stiffness, beyond that provided by curved wire 460, between first segment distal end 164 and second segment proximal end 166. According to some embodiments, the provision of extra stiffness may help in preventing curved wire 460 from being undesirably deformed when switching from the guiding configuration to the work configuration. In particular, when switching from the guiding configuration to the work configuration, mechanical spring 570 may help prevent curved wire proximal end 476 and curved wire distal end 478 from being displaced from longitudinal axis B, or from being too closely pushed toward one another such that curved wire 460 assumes a horseshoe shape.

According to some embodiments, the work element does not include a convex-shaped lesion-smoothing member. FIGS. 6a-6b schematically a side-view of a portion 600 of a guidewire 602 (not depicted in full in the Figures) in a guiding configuration and in a work configuration, respectively. Guidewire 602 provides an alternative embodiment to guidewire 402, in which the work element is not curved, at least not when in the guiding configuration, being straight, or substantially straight, instead. Guidewire 602 differs from guidewire 402 in including a work element 658, in place of work element 158. According to some embodiments, first segment 150, work element 658, and second segment 152 are made of a single piece of wire.

Work element 658 includes a lesion-smoothing member 710 in the form of a wire, a first arm 720 in the form of a wire, and a second arm 730 in the form of a wire. First arm 720 extends from first segment distal end 164 to a proximal end (not numbered) of lesion-smoothing member 710. Second arm 730 extends from a distal end (not numbered) of lesion-smoothing member 710 to second segment proximal end 166. In the guiding configuration, work element 658 is straight or substantially straight (as depicted in FIG. 6a).

Guidewire 602 includes two pairs of notches. A first notch pair 680 includes a first notch 680a and a second notch 680b. A second notch pair 690 includes a third notch 690a and a fourth notch 690b. First notch 680a and second notch 680b each consists of a groove into an upper portion (not numbered) of guidewire 602. First notch 690a and second notch 690b each consists of a groove into a lower portion (not numbered) of guidewire 602. The terms “upper” and “lower”, as used with respect to FIGS. 6a-6b, are relative to an arrow U, which indicates the “upwards” direction. According to some embodiments, as depicted in the FIG. 6a, the notches are triangular.

First notch 680a is located at a joining region 762 of first segment 150 and first arm 720. Second notch 680b is located at a joining region 768 of second arm 730 and second segment 152. First notch 690a is located at a joining region 764 of first arm 720 and lesion-smoothing member 710. Second notch 690b is located at a joining region 766 of lesion-smoothing member 710 and second arm 730.

FIG. 6b schematically depicts guidewire 602 in the work configuration, wherein lesion-smoothing member 710 radially projects relative to longitudinal axis B. Notches 680a, 680b, 690a, and 690b are not visible in FIG. 6b, as the walls (not numbered) of each notch, respectively, are pressed towards one another (that is to say, each of the notches is clamped).

A catheter system including guidewire 602, in particular, a catheter system similar to catheter system 400 except for including guidewire 602 in place of guidewire 402, will be operated essentially similarly to catheter system 400. To switch from the guiding configuration to the work configuration, first segment 150 is distally pushed, when the balloon of the catheter system is anchored within an intraluminal passage and inflated. The distal push forces the walls of each notch, respectively, towards one another. As a result, due to second notch pair 680 being located on the upper portion of guidewire 602, and due to second notch pair 690 being located on the lower portion of guidewire 602, wall-smoothing element 710 is lifted relative to longitudinal axis B.

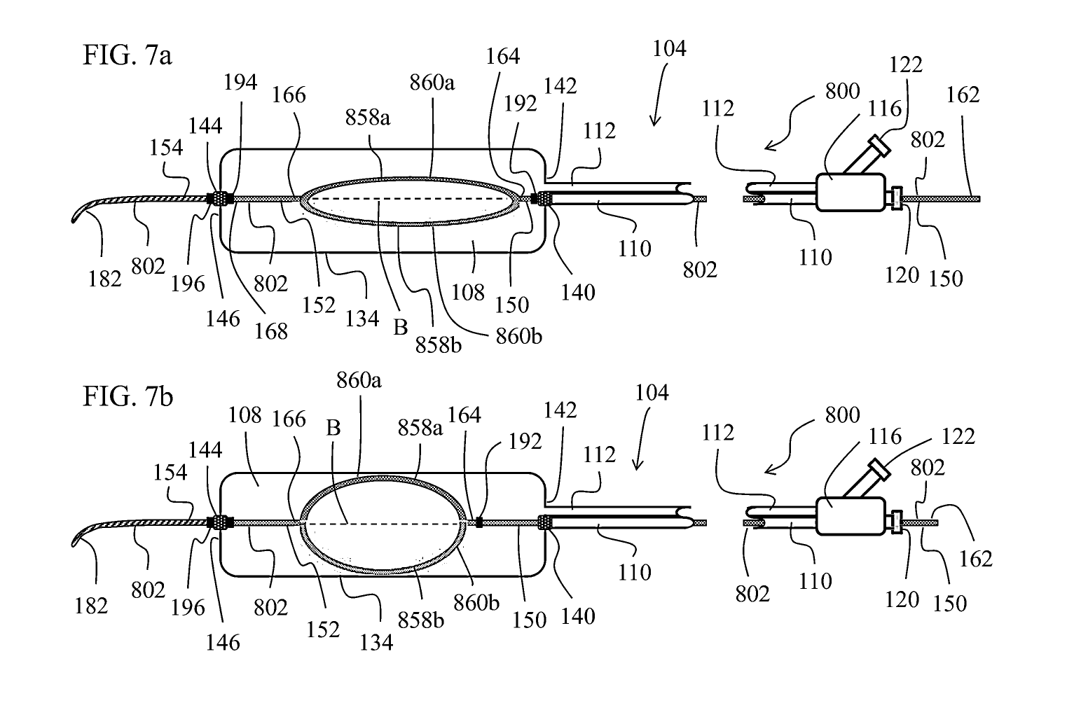

Another exemplary embodiment of a catheter system 800 is schematically depicted in FIGS. 7a-7b. Catheter system 800 is essentially similar to catheter system 400 except for including two work elements instead of one, as explained in the following. Making reference to FIG. 7a, a side-view of a catheter system 800, including catheter body 104 and a guidewire 802, is shown in a guiding configuration, with balloon 108 inflated. Guidewire 802 includes first segment 150, second segment 152, third segment 154, and a first work element 858a and a second work element 858b. Each of work elements 858a and 858b is essentially similar to work element 458, and similarly connected to first segment 150 and to second segment 152. That is to say, first work element 858a is in the form of a first curved wire 860a, which is essentially similar to curved wire 460. First curved wire 860a is connected at the respective ends thereof to first segment distal end 164 and to second segment proximal end 166, respectively. Similarly, second work element 858b is in the form of a second curved wire 860b, which is essentially similar to curved wire 460. Second curved wire 860a is connected at the respective ends thereof to first segment distal end 164 and to second segment proximal end 166, respectively. Curved wires 860a and 860b are symmetrically positioned with respect to longitudinal axis B, opposite to one another.

FIG. 7b schematically depicts a side-view of catheter system 800 in a work configuration. In the work configuration, each of curved wires 860a and 860b radially projects further from longitudinal axis B than in the guiding configuration. That is to say, in the work configuration curved wire 860a and 860b are more curved than in the guiding configuration, essentially as explained above with respect to curved wire 460.

To switch from the guiding configuration to the work configuration, first segment 150 is distally pushed, essentially as described above in the description of catheter system 400. To switch from the working configuration to the guiding configuration, first segment 150 is released, and, according to some embodiments, proximally pulled, essentially as described above in the description of catheter system 400.

It is noted that in operation, e.g. when balloon 108 is inflated and anchored inside a target blood vessel, for every full revolution (rotation) of first segment 150, the vessel inner walls are “swept” twice: once by first curved wire 860a, and once by second curved wire 860b.

Also contemplated are embodiments of a catheter system (not depicted in the Figures) including three or more curved wires, such as a curved wire 460, which may be symmetrically disposed relative to a longitudinal axis of the catheter system, such as longitudinal axis B (e.g. in a catheter system including three symmetrically positioned curved wires, the angle between each adjacent pair of curved wires will equal 120°).

Similarly, also contemplated are embodiments of a catheter system (not depicted in the Figures) including two or more work elements, such as work element 158, which are symmetrically disposed relative to a longitudinal axis of the catheter system (such as longitudinal axis B). According to some such embodiments, wherein the work element includes a convex surface, such as convex surface 210, in the guiding configuration, the plurality of work elements define a substantially closed ellipsoidal surface. According to some such embodiments, wherein the work element includes a convex surface, such as convex surface 210, in the work configuration too the plurality of work elements define a substantially closed ellipsoidal surface. In such embodiments, in the guiding configuration, the outer face of each convex surface is partially covered by the inner face of a respective one of the two convex surfaces adjacent thereto (e.g. the convex surface located clockwise thereto).

According to some embodiments, the work element is an elliptical, ellipsoidal, or rounded body, which may be rigid or resiliently flexible.

As used herein, “proximal segment” with respect to a guidewire, such as any one of guidewires 102, 402, 502, 602, and 802, may be used interchangeably with “first segment” (e.g. first segment 150). “Distal segment” with respect to a guidewire, such as any one of guidewires 102, 402, 502, 602, and 802 may be used to refer to the totality of “second segment” and “third segment” (e.g. second segment 152 and third segment 154 make up the distal segment of guidewire 102). “Non-exposed segment” with respect to a guidewire, such as any one of guidewires 102, 402, 502, 602, and 802 may be used interchangeably with “second segment” (e.g. second segment 152). “Exposed segment” with respect to a guidewire, such as any one of guidewires 102, 402, 502, 602, and 802 may be used interchangeably with “third segment” (e.g. third segment 154).

As used herein, “flexible” and “resiliently flexible” with respect to a guidewire, such as any one of guidewires 102, 402, 502, 602, and 802, and components thereof (e.g. convex surface 210 of guidewire 102 and curved wire 460 of guidewire 402) may be used interchangeably.

According to some embodiments, there is provided a catheter system for intraluminal passages, comprising:
a guidewire, the guidewire including a first segment, a second segment, and a work element extending from the first segment to the second segment;
a tube having a proximal tube end and a distal tube end, being flexible and mounted on said guidewire such that said guidewire extends therethrough; and
an inflatable balloon having an inner surface and an outer surface disposed at the distal end of the tube, the inflatable balloon having a proximal balloon end mounted on said first segment, and a distal balloon end mounted on said second segment, thereby enveloping the work element;

wherein said work element comprises an elongated, convex member projecting radially from a longitudinal axis of said inflatable balloon and mechanically associated on a first convex member end with said first segment and on a second convex member end with said second segment; and

wherein said work element is configured to allow for controllable rotation about said longitudinal axis, when said balloon is inflated with a fluid and is inside an intraluminal passage, such that the convex member rotates along the inner surface of said inflatable balloon and simultaneously pushes against said inner surface.

Although certain aspects of the invention have been exemplified in the context of treating blockages in blood vessels, it will be readily appreciated by a person skilled in the art that these same aspects may also be of relevance in treating blockages in intraluminal passages other than blood vessels, such as ureteral passages.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. No feature described in the context of an embodiment is to be considered an essential feature of that embodiment, unless explicitly specified as such.

Although steps of methods according to some embodiments may be described in a specific sequence, methods of the invention may comprise some or all of the described steps carried out in a different order. A method of the invention may comprise all of the steps described or only a few of the described steps. No particular step in a disclosed method is to be considered an essential step of that method, unless explicitly specified as such.

Although the invention is described in conjunction with specific embodiments thereof, it is evident that numerous alternatives, modifications and variations that are apparent to those skilled in the art may exist. Accordingly, the invention embraces all such alternatives, modifications and variations that fall within the scope of the appended claims. It is to be understood that the invention is not necessarily limited in its application to the details of construction and the arrangement of the components and/or methods set forth herein. Other embodiments may be practiced, and an embodiment may be carried out in various ways.

The phraseology and terminology employed herein are for descriptive purpose and should not be regarded as limiting. Citation or identification of any reference in this application shall not be construed as an admission that such reference is available as prior art to the invention.

Section headings are used herein to ease understanding of the specification and should not be construed as necessarily limiting.

1. A catheter system, comprising:
a guidewire comprising a proximal segment, a distal segment, and at least one work element, being elongated and extending therebetween from a distal end of said proximal segment to a proximal end of said distal segment;
a tube, being flexible and mounted on said guidewire such that said proximal segment longitudinally extends therethrough; and
a balloon, being inflatable and enveloping said at least one work element, said balloon being connected at a balloon proximal end to said tube at a distal end thereof;
wherein said guidewire is controllably switchable between a guiding configuration, for maneuvering the catheter system through intraluminal passages, and a work configuration, in which said at least one work element radially projects relative to a longitudinal axis of said balloon and pushes against a surface of said balloon when said balloon is inflated with fluid and anchored in an intraluminal passage; and wherein said at least one work element is non-expandable.

2. The catheter system of claim 1, wherein said at least one work element comprises a lesion-smoothing member, said lesion-smoothing member being a rigid or resiliently flexible surface or a rigid or resiliently flexible wire.

3. The catheter system of claim 2, wherein in the guiding configuration said at least one work element substantially does not project relative to the longitudinal axis.

4. The catheter system of claim 2, wherein said guidewire is continuously switchable between the guiding configuration and the work configuration, thereby allowing to control the amount of projection of said at least one work element and the amount of force exerted by said at least one work element against said balloon surface when said balloon is inflated with a fluid and anchored in an intraluminal passage.

5. The catheter system of claim 4, wherein said at least one work element is further configured to allow for controllable rotation about the longitudinal axis, such as to allow said lesion-smoothing member to slide along said balloon surface and simultaneously push there against when said guidewire is in the work configuration and said balloon is inflated with a fluid and anchored in an intraluminal passage; and/or
wherein said at least one work element is further configured to allow for reciprocal motion, such as to allow said lesion-smoothing member to substantially proximally and distally slide along said balloon surface and simultaneously push there against when said guidewire is in the work configuration and said balloon is inflated with a fluid and anchored in an intraluminal passage.

6. The catheter system of claim 5, wherein said at least one work element is configured to allow for controllable rotation about the longitudinal axis, the controllable rotation being effected by rotating said proximal segment.

7. The catheter system of claim 6, wherein the mounting of said balloon distal end on said distal segment is such as to prevent any proximal and distal motion of said balloon distal end relative to said distal segment.

8. The catheter system of claim 7, further configured to allow switching from the guiding configuration to the work configuration by distally pushing said proximal segment when said balloon is anchored.

9. The catheter system of claim 8, wherein said distal segment comprises an exposed segment and a non-exposed segment, located outside of said balloon and within said balloon, respectively, said exposed segment comprising a pliable tip at a distal end thereof.

10. The catheter system of claim 9, wherein said tube distal end comprises a first bearing mounted thereon and connected to said balloon proximal end, said proximal segment passing through said first bearing; and wherein said distal segment comprises a second bearing mounted thereon and connected to said balloon distal end, said first bearing and said second bearing being configured to allow rotating said guidewire without rotating said tube and said balloon, said first bearing being further configured to allow proximal and distal motion therethrough of said proximal segment.

11. The catheter system of claim 10, wherein said proximal segment comprises a first disc, mounted perpendicularly thereto and distally relative to said first bearing such as to be positioned adjacent thereto when said guidewire is in the guiding configuration, and wherein said distal segment comprises a second disc and a third disc, mounted perpendicularly thereto and proximally and distally relative to said second bearing, respectively, such as to be positioned adjacent thereto.

12. The catheter system of claim 11, wherein said exposed segment and said non-exposed segment are mechanically associated via a ratchet within said second bearing, said ratchet being configured to allow for (i) joint rotation of said exposed segment together with said non-exposed segment, said at least one work element, and said proximal segment when said proximal segment is rotated in one sense, and (ii) rotation only of said non-exposed segment, said at least one work element, and said proximal segment when said proximal segment is rotated in the other sense.

13. The catheter system of claim 12, wherein said lesion-smoothing member is substantially convex.

14. The catheter system of claim 13, wherein said lesion-smoothing member is a convex surface, and wherein said at least one work element further comprises a first arm, mechanically associating said convex surface on a proximal end thereof with said distal end of said proximal segment, and a second arm, mechanically associating said convex surface on a distal end thereof with said proximal end of said distal segment.

15. The catheter system of claim 14, further comprising a first joining region connecting said proximal segment distal end to said first arm, a second joining region connecting said first arm to said convex surface proximal end, a third joining region connecting said convex surface distal end to said second arm, and a fourth joining region connecting said second arm to said distal segment proximal end;
wherein said joining regions comprise hinges, or said joining regions are more flexible than said proximal segment, said first arm, said convex surface, said second arm, and said distal segment, such as to allow said convex surface to be lifted when said proximal segment is pushed in the distal direction when said balloon is inflated with a fluid and anchored within an intraluminal passage.

16. The catheter system of claim 15, further comprising a duct fluidly connected at a distal end thereof to said balloon and configured to be coupled, at a proximal end thereof, to a fluid source or vacuum, said duct being longitudinally joined at least at a distal portion thereof to said tube.

17. The catheter system of claim 16, configured to allow for the controllable rotation to be effected by a motor and/or manually.

18. The catheter system of claim 5, wherein the intraluminal passage is a blood vessel.

19. The catheter system of claim 5, comprising a plurality of said work element, said plurality of work elements being symmetrically disposed about the longitudinal axis of said balloon.

20. A method for treating blockage in intraluminal passages, comprising the steps of:
introducing a catheter system, according to claim 1, into an intraluminal passage in a body of a subject, the catheter system being in the guiding configuration, and maneuvering the catheter system until the balloon reaches a location of a target lesion in the intraluminal passage, with the work element facing the target lesion;
inflating the balloon with a fluid until the balloon pushes against inner walls of the intraluminal passage, thereby anchoring the balloon;
switching the catheter system into the work configuration, thereby pushing the work element against the target lesion and compressing the target lesion.
